(19) United States
(12) Patent Application Publication
(10) Pub. No.: US 2026/0242670 A1
(43) Pub. Date: Aug. 20, 2026

(54) Carbon Dioxide Hydrogenation to Produce Fuels Using Petroleum Refinery Facilities

(71) Applicant: Chevron U.S.A. Inc., San Ramon, CA (US)

(72) Inventors: Cesar Francisco Ovalles, Walnut Creek, CA (US); Evan Michael Visser, Spring, TX (US); William Richard Boorujy, Livermore, CA (US)

(21) Appl. No.: 19/543,097

(22) Filed: Feb. 18, 2026

Related U.S. Application Data
(60) Provisional application No. 63/759,853, filed on Feb. 18, 2025.

(51) Int. Cl. C10G 2/00; C07C 1/12; C10G 61/02; C10G 67/02

(52) U.S. Cl. CPC C10G 2/50; C07C 1/12; C10G 61/02; C10G 67/02; C10G 2400/02; C10G 2400/04; C10G 2400/08

(57) ABSTRACT

A process for direct or indirect hydrogenation of CO₂ to produce fuels and fuel components utilizing existing downstream facilities used to produce oil and gas, for example petroleum refineries, is provided. The hydrogenation process typically uses CO₂ separated from a source at the petroleum refinery and also low carbon intensity (CI) hydrogen. The process is able to produce low carbon-intensity gasoline, diesel, sustainable aviation fuel, and/or other low carbon intensity fuel components. Gases generated from the hydrogenation step can be recycled and re-used in the hydrogenation process. An aqueous phase produced from the hydrogenation step can be disposed of using an existing water treatment plant at the refinery.

### CROSS REFERENCE TO RELATED APPLICATIONS

This application claims the benefit of U.S. Provisional Application No. 63/759,853, filed Feb. 18, 2025, the disclosure of which is incorporated herein by reference in its entirety.

### TECHNICAL FIELD

This disclosure relates to a process for direct or indirect hydrogenation of carbon dioxide.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Carbon capture, utilization, and storage (CCUS) is generally a process intended to reduce the level of carbonaceous gases (most commonly carbon dioxide (CO₂)) within the atmosphere. This may include the capture of carbonaceous gases from sources of emissions so that they do not enter the atmosphere, or to directly remove the carbonaceous gases from the atmosphere. In some instances, the captured gases may be used in other processes, such as in the production of new materials, chemicals, and so forth, or as a process gas. Alternatively, the captured carbonaceous gases may be stored or sequestered in a variety of ways, such as through chemical transformation and/or injection into a geological formation.

Carbon capture, utilization, and storage (CCUS) in general may afford operators in the oil and gas industry to enhance efficiencies while also reducing carbon intensity in their operations. However, only a small fraction of the global carbon dioxide (CO₂) emitted yearly is captured and utilized, and most of the CO₂ transformation concepts available in the market are in their infancy or are not economical.

Carbon intensity (CI) is a measure of carbon dioxide and other greenhouse gases that are emitted or released. Carbon intensity (CI) can be defined as grams of CO₂ equivalent (gCO₂e) per unit of energy, for example a megajoule (MJ). In other words, the carbon intensity of a product may be given in units of grams of CO₂ equivalent per megajoule of product (gCO₂e/MJ of product), since products have a calorific value. The conversion of CO₂ to a low carbon intensity (CI) fuel can be conducted via an indirect or direct route. In the indirect route, the reaction of CO₂ with H₂ takes place via the reverse water-gas shift reaction (RWGS) to generate syngas (H₂/CO), followed by the Fischer-Tropsch process to produce hydrocarbons (equation 1) that can be used as fuels or a component of fuel.

CO₂+H₂→Syngas (H₂/CO)→Hydrocarbons+H₂O (1)

The indirect route described by equation 1 is relatively complex and capital intensive due to the need for syngas generation and product upgrading from a wide product distribution. For example, Peter et al. studied the coupling of Fischer-Tropsch synthesis to produce synthetic diesel and kerosene with a high-temperature electrolysis unit. See Peters, R., Wegener, N., Samsun, R. C., Schorn, F., Riese, J., Grünewald, M., Stolten, D., “A Techno-Economic Assessment of Fischer-Tropsch Fuels Based on Syngas from Co-Electrolysis”, Processes 2022, 10, 699. In this work, it was found that converting CO₂ to low carbon intensity fuels via indirect routes (synthesis gas) requires the produced hydrocarbons to be dehydrated, separated, hydro-treated, and reformed, increasing the process' complexity, operating expenses, and capital expenses.

A similar downstream process with high complexity has been found by Meurer and Kern, who studied the Fischer-Tropsch synthesis for sustainable kerosene production. See Meurer, A.; Kern, J., “Fischer-Tropsch Synthesis as the Key for Decentralized Sustainable Kerosene Production.” Energies 2021, 14, 1836. In this process, after the Fischer Tropsch reactor, the separation process becomes very complex with four flash drums, one hydrocracker, as well as several heat exchangers. Other authors have reported similarly complicated separation processes during their studies of RWGS and FT processes. See Medrano-García, J. D., Charalambous, M. A., Guillén-Gosálbez, G., “Economic and Environmental Barriers of CO2-Based Fischer-Tropsch Electro-Diesel”, ACS Sustainable Chem. Eng. 2022, 10, 11751-11759; and Rodríguez Vallejo, D. F., de Klerk, A., “Improving the Interface between Fischer-Tropsch Synthesis and Refining’, Energy & Fuels, 2013, 27, 3137-3147. Thus, transforming CO₂ to low carbon intensity fuels via syngas-based routes requires that the produced kerosene be dehydrated, separated, and hydrocracked. These complexities reduce the profitability of the process.

Alternatively, direct routes for conversion of CO₂ to a low carbon intensity (CI) fuel have been discovered. One route is direct hydrogenation of CO₂ to produce fuels and can be summarized by Equation 2.

CO₂+H₂→Hydrocarbons+H₂O (2)

This direct process uses a single reactor, a multifunctional metal catalyst system, and is operated at optimized reaction conditions to generate syngas in-situ with C5+ selectivities in the range of 60-80%. See Chen, C., Garedew, M., Sheehan, S. W., “Single-Step Production of Alcohols and Paraffins from CO2 and H2 at Metric Ton Scale”, ACS Energy Lett. 2022, 7, 988-992; and Benzhen Yao, B., Xiao, T., Makgae, O. A., Jie, X., Gonzalez-Cortes, S., Guan, S., Kirkland, A. I., Dilworth, J. R., Al-Megren, H. A., Alshihri, S. M., Dobson, P. J., Owen, G. P., Thomas, J. M., Edwards, P. P. “Transforming carbon dioxide into jet fuel using an organic combustion-synthesized Fe—Mn—K catalyst” Nat. Commun., (2020) 11:6395-6406. Thus, a multistep process could be envisioned to dehydrate, separate, hydro-treat, and isomerize the products to fuel, with concomitant increases in capital and operating costs. As in the indirect route (Equation 1), the direct CO₂ hydrogenation route (Equation 2) suffers similar disadvantages in terms of the high downstream complexity associated with separating and stabilizing the reaction products. Thus, it is now recognized that a reduction in capital and operating costs associated with direct and indirect hydrogenation of CO₂ to produce fuels is desired.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

One aspect of the disclosure provides a process for producing fuel or a fuel component having a low carbon intensity (CI). The process can be conducted at an existing downstream facility used to produce oil or gas, such as a petroleum refinery, in order to reduce costs and reduce emissions. The process includes hydrogenating carbon dioxide (CO₂) at the downstream facility to produce gases, hydrocarbons, and an aqueous phase. The process further includes separating, processing, and/or recycling the gases, the hydrocarbons, and the aqueous phase at the downstream facility.

The CO₂ used in the hydrogenation step may come from any number of sources, and in certain embodiments may be obtained from a source located at the downstream facility, for example flue gas. According to some embodiments, the hydrogenation step may including reacting the CO₂ which hydrogen that has a carbon intensity of not greater than 50 g CO₂e/MJ, for example a carbon intensity (CI) ranging from 11 gCO₂e/MJ to 50 gCO₂e/MJ.

The hydrogenation step can include indirect hydrogenation of the carbon dioxide according to Equation 1: CO₂+H₂→Syngas (H₂/CO)→Hydrocarbons+H₂O, or direct hydrogenation of the carbon dioxide according to Equation 2: CO₂+H₂→Hydrocarbons+H₂O.

The gases produced by the process may include CO₂, CO, and H₂. The products of the hydrogenation step typically have a carbon intensity of not greater than 80 gCO₂e/MJ. The process may include processing the gases using equipment at the downstream facility; and/or recycling the gases and using the gases in the downstream facility.

According to example embodiments, the process may include separating the gases, the hydrocarbons, and the aqueous phase from one another; processing the gases using equipment at the downstream facility; and recycling the gases and using the gases in the downstream facility. For example, the process may include feeding the aqueous phase to a water treatment plant at the downstream facility for disposal. The process may also include feeding the hydrocarbons to a crude distillation unit and distilling the hydrocarbons to produce a feedstock. The process can also include dividing the hydrocarbons into naphtha, jet fuel, diesel, vacuum gas oil (VGO), and/or waxes. The naphtha can include C5-C10 hydrocarbons, and the process can include feeding the naphtha to a naphtha hydrotreater to produce a gasoline component. The jet fuel may include C10-C15 hydrocarbons, and the process can include feeding the jet fuel to a jet fuel hydrotreater to produce a jet fuel component. The diesel can include C15-C23 hydrocarbons, and the process can include feeding the diesel to a diesel hydrotreater to produce a diesel component. The vacuum gas oil can include C23+ hydrocarbons, and the process can include feeding the vacuum gas oil to a fluid catalytic cracker or another hydrocracker within the downstream facility to produce motor gasoline (MOGAS), jet fuel, diesel and/or fuel gas. The wax can include C19-C24 hydrocarbons with the formula CₙH₂ₙ₊₂, and the process can include processing the vacuum gas oil and the wax in a fluid catalytic cracker or another hydrocracker within the downstream facility to produce motor gasoline (MOGAS), jet fuel, diesel and/or fuel gas. The process could also include distilling the naphtha and diesel in a coker distillation unit, and hydro-treating the naphtha and diesel.

### BRIEF DESCRIPTION OF THE DRAWINGS

The drawings described herein are for illustrative purposes only of selected embodiments and are not intended to limit the scope of the present disclosure. The inventive concepts associated with the present disclosure will be more readily understood by reference to the following description in combination with the accompanying drawings, wherein:

FIG. 1 is a process flow diagram showing production of a low carbon intensity (CI) hydrocarbon via direct hydrogenation of CO₂ using existing petroleum refinery facilities according to an embodiment of this disclosure;

FIG. 2 is a process flow diagram showing the production of a low carbon intensity (CI) hydrocarbon via indirect hydrogenation of CO₂ using existing petroleum refinery facilities according to an embodiment of this disclosure;

FIG. 3 is a process flow diagram showing production of a low carbon intensity (CI) naphtha, jet fuel, and diesel via direct hydrogenation of CO₂ using existing petroleum refinery facilities according to an embodiment of this disclosure;

FIG. 4 is a process flow diagram showing production of a low carbon intensity (CI) naphtha, jet fuel, and diesel via indirect hydrogenation of CO₂ using existing petroleum refinery facilities according to an embodiment of this disclosure;

FIG. 5 is a process flow diagram showing production of a low carbon intensity (CI) hydrocarbon via direct hydrogenation of CO₂ using existing petroleum refinery facilities according to an embodiment of this disclosure; and

FIG. 6 is a process flow diagram showing the production of a low carbon intensity (CI) hydrocarbon via indirect hydrogenation of CO₂ using existing petroleum refinery facilities according to an embodiment of this disclosure.

### DETAILED DESCRIPTION

The disclosure generally relates to a process for the production of low carbon intensity (CI) fuels and/or fuel components via the direct or indirect hydrogenation of CO₂. In particular, the disclosed processes utilize existing downstream facilities, for example equipment and space available at petroleum refineries to separate, hydrotreat, store, and/or blend hydrocarbon products. The process may have several advantages, including for example a reduction in the capital and operating costs typically associated with CO₂ hydrogenation and a decrease in greenhouse gas emissions. Specifically, the process may generate low carbon intensity (CI) fuels such as gasoline, diesel, sustainable aviation fuel, and/or fuel components.

As stated above, the process can include the indirect or direct hydrogenation of CO₂. According to the indirect route, the reaction of CO₂ with H₂ takes place via a reverse water-gas shift reaction (RWGS) to generate syngas (H₂/CO), followed by the Fischer-Tropsch process to produce hydrocarbons, which can be used as fuel or a component of fuel. The indirect process can be summarized by Equation 1.

CO₂+H₂→Syngas (H₂/CO)→Hydrocarbons+H₂O (1)

The direct hydrogenation of CO₂ to produce fuels can be summarized by Equation 2.

CO₂+H₂→Hydrocarbons+H₂O (2)

The direct process may include a single reactor and a multifunctional metal catalyst system. The direct process is operated at optimized reaction conditions to generate syngas in-situ with C5+ selectivities in the range of 60-80%.

The CO₂ feedstock used in the CO₂ hydrogenation process can be obtained from various sources but is preferably a byproduct of the refinery. This provides for a reduction in capital and operating expenditures associated with the CO₂ hydrogenation unit and process, thus increasing profitability. In addition, the hydrogenation step includes reacting the CO₂ with low carbon intensity hydrogen, for example hydrogen having a carbon intensity of not greater than 50 gCO₂e/MJ, for example a carbon intensity (CI) ranging from 11 gCO₂e/MJ to 50 gCO₂e/MJ.

The hydrogenation step produces gases, hydrocarbons, and an aqueous phase; and the gases, the hydrocarbons, and the aqueous phase can be separated, processed, and/or recycled at the downstream facility. The process can take advantage of existing downstream facilities, for example facilities available at petroleum refineries, to separate, hydrotreat, store, and/or blend hydrocarbon products. There is significant integration between the refinery and CO₂ hydrogenation unit. For example, the processing could including hydrocracking, hydrotreating, storage, blending, distillation, and fluid catalytic cracking, all using components available at the existing downstream facilities. Water disposal and gas processing units at the refinery can also be utilized, leading to economic advantages. Existing transportation systems and storage units located at the downstream facility can also be used. For example, loading racks, pipelines, laboratory systems could be used. Integrating the process within an existing refinery can reduce the need to build new tanks and blending units. Another advantages of the process according to the present disclosure is the ability to switch traditional feeds to “renewable” feeds and also improve traceability of the feed source. Due to the integration of the process in the existing refinery, it is easier to figure out how much of the product is made from a renewable feed.

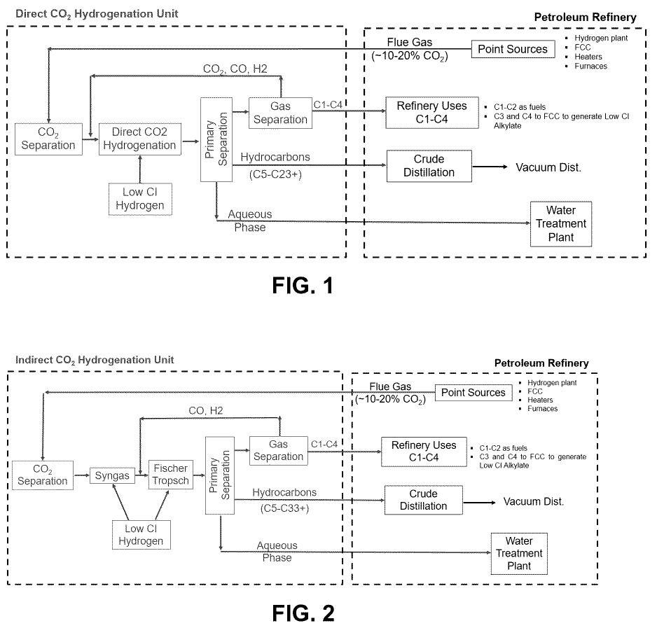

Referring now to FIG. 1, an example embodiment of in integration scheme in accordance with present embodiments is shown by way of a process flow diagram. In particular, in FIG. 1, a direct CO₂ hydrogenation unit is integrated with various equipment within an existing petroleum refinery. The direct CO₂ hydrogenation unit performs direct hydrogenation of CO₂ according to Equation 2 using a metal catalyst including nickel, palladium, and/or platinum.

The CO₂ may be separated from a source at the existing petroleum refinery, or may be from a source outside of the refinery. By way of non-limiting example, the CO₂ can be obtained from flue gas containing 10% to 20% CO₂ from a hydrogen plant, fluid catalytic cracking unit, heater, or furnace. The direct hydrogenation step occurs at this same petroleum refinery. The direct hydrogenation step according to certain embodiments may also use low carbon intensity (CI) hydrogen, for example green hydrogen, which has a carbon intensity (CI) of 11 gCO₂e/MJ or blue hydrogen (e.g., hydrogen obtained by steam methane reforming). The hydrogenation step generates hydrocarbons in gas phase (C1-C4) and liquid phase (C5-C23+), and also generates an aqueous phase product. Example catalysts include nickel, palladium, and platinum.

Next, according to this example embodiment, the stream undergoes a primary separation step and is divided into the gases (CO₂, CO, H₂, and C1-C4), the hydrocarbons (C5-C23+), and the aqueous phase. The aqueous phase is composed of water (>95% wt. %) with small amounts of alcohols (<3 wt. %), acetones (<2 wt. %), aldehydes (<1 wt. %), and hydrocarbons (<1 wt. %). The gases resulting from the primary separation step are divided. The CO₂, CO, and H₂ are processed using existing equipment at the downstream facility. These gases are recycled and can be used in the hydrogenation step. The C1 to C4 gases can be used in other areas of the refinery. For example, the C1-C2 gases can be used as fuels, and the C3 and C4 gases can serve as feedstocks to generate low carbon intensity (CI) alkylate. The hydrocarbons resulting from the primary separation step are fed to a crude distillation unit, typically undergo vacuum distillation, and can be used as refinery feedstock. The use of sustainable CO₂ as a carbon source to produce the low carbon intensity hydrocarbon decreases the carbon intensity of the facility, and at the same time, higher volumes of crude oil feedstocks can be processed in the distillation unit. The aqueous phase is disposed of using an existing water treatment plant at the refinery. There is no need for a costly separation process for the CO₂ hydrogenation, thus decreasing costs and increasing profitability.

A second embodiment of the process is shown in FIG. 2 by way of a process flow diagram. This process includes the indirect hydrogenation of CO₂ according to Equation 1 using CO₂ separated from a source of CO₂ at an existing petroleum refinery, for example a hydrogen plant, fluid catalytic cracking unit, heater, or furnace. The indirect hydrogenation step occurs at this same petroleum refinery. The indirect hydrogenation step according to the second embodiment also uses low carbon intensity (CI) hydrogen, for example green hydrogen, blue hydrogen, or hydrogen obtained by steam methane reforming using renewable natural gas. Unlike the direct hydrogenation process of the first example embodiment, the indirect process of the second embodiment generates syngas from the CO₂ and low carbon intensity hydrogen, which is further reacted over a Fischer-Tropsch catalyst to yield the hydrocarbons with a carbon distribution between C1 and C33+. The indirect hydrogenation also generates gases and an aqueous product.

Next, according to the second example embodiment, the stream undergoes a primary separation step and is divided into the gases (CO₂, CO, H₂, and C1-C4), the hydrocarbons (C5-C23+), and the aqueous phase. The gases resulting from the primary separation step are separated. The CO₂, CO, and H₂ is recycled, and the C1 to C4 gases are used in other areas of the refinery. For example, the C1-C2 gases can be used as fuels, and the C3 and C4 gases can serve as feedstocks to generate low carbon intensity (CI) alkylate. The gases are processed using existing equipment at the downstream facility. The hydrocarbons resulting from the primary separation step are fed to a crude distillation unit, typically undergo vacuum distillation, and can be used as refinery feedstock. The use of sustainable CO₂ as a carbon source to produce the low carbon intensity hydrocarbon decreases the carbon intensity of the facility, and at the same time, higher volumes of crude oil feedstocks can be processed in the distillation unit. The aqueous phase is disposed of using the existing water treatment plant at the refinery. There is no need for a costly separation process for the CO₂ hydrogenation, thus decreasing cost and increasing profitability.

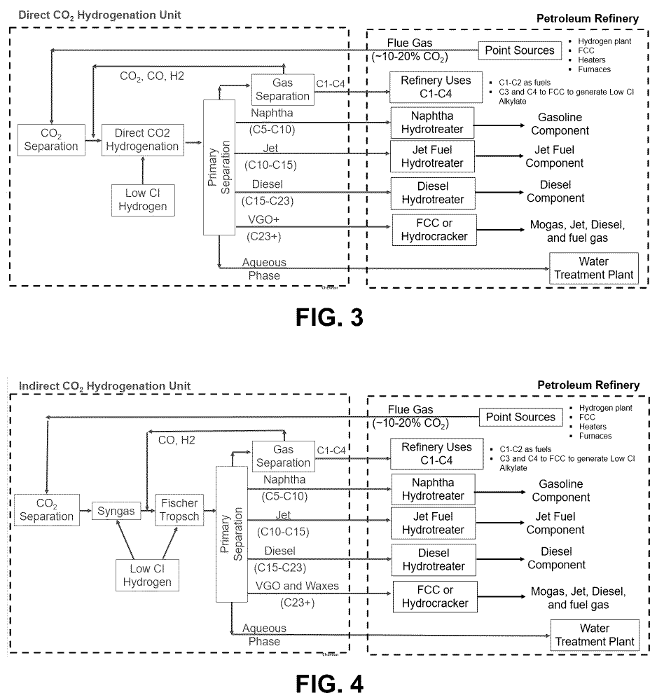

A third embodiment of the process is shown in FIG. 3 by way of a process flow diagram. This process includes the direct hydrogenation of CO₂ according to Equation 2 using CO₂ separated from a source at an existing petroleum refinery. For example, the CO₂ can be obtained from flue gas containing 10% to 20% CO₂ from a hydrogen plant, fluid catalytic cracking unit, heater, or furnace. The direct hydrogenation step occurs at this same petroleum refinery. The direct hydrogenation step according to the third embodiment also uses low carbon intensity (CI) hydrogen, for example green hydrogen, blue hydrogen, or hydrogen obtained by steam methane reforming using renewable natural gas. The hydrogenation step generates gases, hydrocarbons, and an aqueous product using a metal catalyst.

Next, according to the third example embodiment, the stream undergoes a primary separation step and is divided into the gases (CO₂, CO, H₂, and C1-C4), the hydrocarbons (C5-C23+), and an aqueous phase. The CO₂, CO, and H₂ is recycled, and the C1 to C4 gases are used in other areas of the refinery. For example, the C1-C2 gases can be used as fuels, and the C3 and C4 gases can serve as feedstocks to generate low carbon intensity (CI) alkylate. The aqueous phase is disposed of using the existing water treatment plant at the refinery. There is no need for a costly separation process for the CO₂ hydrogenation, thus decreasing cost and increasing profitability.

Unlike the first and second embodiments, the hydrocarbons resulting from the primary separation step are divided into naphtha, jet fuel, diesel, vacuum gas oil (VGO), and waxes. The naphtha includes C5-C10 hydrocarbons and can be fed to a naphtha hydrotreater to produce a gasoline component. The jet fuel includes C10-C15 hydrocarbons and can be fed to a jet fuel hydrotreater to produce a jet fuel component. The diesel includes C15-C23 hydrocarbons and can be fed to a diesel hydrotreater to produce a diesel component. The vacuum gas oil includes C23+ hydrocarbons, and the wax includes C19-C24 hydrocarbons with the formula CₙH₂ₙ₊₂. The vacuum gas oil and the wax can be processed in a fluid catalytic cracker or another hydrocracker available within the refinery to produce motor gasoline (MOGAS), jet fuel, diesel and/or fuel gas. All of the resulting streams have a low carbon intensity (CI) and can be used as fuel components. The carbon intensity (CI) of the facility is decreased by using sustainable CO₂ as a carbon source, and high-value products are generated.

A fourth embodiment of the process is shown in FIG. 4 by way of a process flow diagram. This process is similar to the process of the third embodiment, except indirect (rather than direct) hydrogenation of CO₂ according to Equation 1 is used to produce low carbon intensity naphtha, jet fuel, and diesel. Again, the CO₂ is separated from a source at an existing petroleum refinery. The indirect hydrogenation step occurs at this same petroleum refinery. For example, the CO₂ can be obtained from flue gas containing 10% to 20% CO₂ from a hydrogen plant, fluid catalytic cracking unit, heater, or furnace. The indirect hydrogenation step according to the fourth embodiment also uses low carbon intensity (CI) hydrogen, for example green hydrogen, blue hydrogen, or hydrogen obtained by steam methane reforming using renewable natural gas. Unlike the direct hydrogenation process of the third embodiment, the indirect process of the fourth embodiment includes the generation of syngas from the CO₂ and low carbon intensity (CI) hydrogen, which is further reacted over a Fischer-Tropsch catalyst to yield the hydrocarbons with a carbon distribution between C1 and C33+. The indirect hydrogenation also generates gases and an aqueous product.

Next, the stream undergoes a primary separation step and is divided into the gases (CO₂, CO, H₂, and C1-C4), the hydrocarbons (C5-C23+), and the aqueous phase. The gases resulting from the primary separation step are divided. The CO₂, CO, and H₂ are recycled, and the C1 to C4 gases are used in other areas of the refinery. For example, the C1-C2 gases can be used as fuels, and the C3 and C4 gases can serve as feedstocks to generate low carbon intensity (CI) alkylate. The aqueous phase is disposed of using the existing water treatment plant at the refinery. There is no need for a costly separation process for this CO₂ hydrogenation process, thus decreasing cost and increasing profitability.

Like the process of the third embodiment, the hydrocarbons resulting from the primary separation step are divided into naphtha, jet fuel, diesel, vacuum gas oil (VGO), and waxes. The naphtha includes C5-C10 hydrocarbons and can be fed to a naphtha hydrotreater to produce a gasoline component. The jet fuel includes C10-C15 hydrocarbons and can be fed to a jet fuel hydrotreater to produce a jet fuel component. The diesel includes C15-C23 hydrocarbons and can be fed to a diesel hydrotreater to produce a diesel component. The vacuum gas oil includes C23+ hydrocarbons, and the wax includes C19-C24 hydrocarbons with the formula CₙH₂ₙ₊₂. The vacuum gas oil and the wax can be processed in a fluid catalytic cracker or another hydrocracker available within the refinery to produce motor gasoline (MOGAS), jet fuel, diesel and/or fuel gas. All of the resulting streams have a low carbon intensity (CI) and can be used as fuel components. The carbon intensity (CI) of the facility is decreased by using sustainable CO₂ as a carbon source, and high-value products are generated.

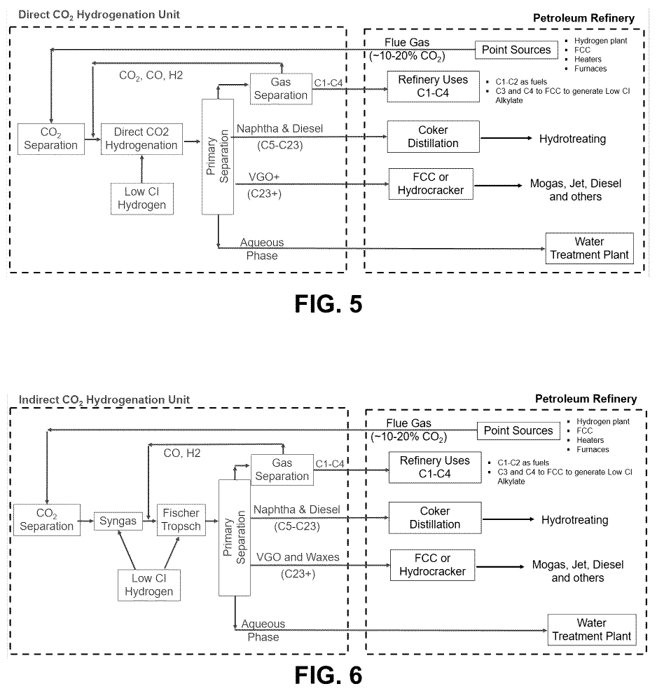

A fifth embodiment of the process is shown in FIG. 5 by way of a process flow diagram. This process includes the direct hydrogenation of CO₂ according to Equation 2 using CO₂ separated from a source at an existing petroleum refinery. For example, the CO₂ can be obtained from flue gas containing 10% to 20% CO₂ from a hydrogen plant, fluid catalytic cracking unit, heater, or furnace. The direct hydrogenation step occurs at this same petroleum refinery. The direct hydrogenation step according to the fifth embodiment also uses low carbon intensity (CI) hydrogen, for example green hydrogen, blue hydrogen, or hydrogen obtained by steam methane reforming using renewable natural gas. The hydrogenation step generates gases, hydrocarbons, and an aqueous product using a metal catalyst.

Next, according to the fifth example embodiment, the stream undergoes a primary separation step and is divided into the gases (CO₂, CO, H₂, and C1-C4), the hydrocarbons (C5-C23+), and the aqueous phase. The gases resulting from the primary separation step are divided. The CO₂, CO, and H₂ are recycled, and the C1 to C4 gases are used in other areas of the refinery. For example, the C1-C2 gases can be used as fuels, and the C3 and C4 gases can serve as feedstocks to generate low carbon intensity (CI) alkylate. The aqueous phase is disposed of using the existing water treatment plant at the refinery. There is no need for a costly separation process for this CO₂ hydrogenation, thus decreasing cost and increasing profitability.

The hydrocarbons resulting from the primary separation step are divided into naphtha, diesel, and vacuum gas oil (VGO). The naphtha includes C5-C10 hydrocarbons and the diesel includes C15-C23 hydrocarbons. A stream containing naphtha and diesel can be fed to a coker distillation step and hydro-treatment step. The vacuum gas oil includes C23+ hydrocarbons and is fed to a fluid catalytic cracker or another hydrocracker available within the refinery to produce motor gasoline (MOGAS), jet fuel, diesel and/or another fuel. All of the resulting streams have a low carbon intensity (CI) and can be used as fuel components. The carbon intensity (CI) of the facility is decreased by using sustainable CO₂ as a carbon source, and high-value products are generated.

A sixth embodiment of the process is shown in FIG. 6 by way of a process flow diagram. This process includes the indirect hydrogenation of CO₂ according to Equation 1 using CO₂ separated from a source at an existing petroleum refinery. For example, the CO₂ can be obtained from flue gas containing 10% to 20% CO₂ from a hydrogen plant, fluid catalytic cracking unit, heater, or furnace. The indirect hydrogenation step occurs at this same petroleum refinery. The indirect hydrogenation step also uses low carbon intensity (CI) hydrogen, for example green hydrogen, blue hydrogen, or hydrogen obtained by steam methane reforming using renewable natural gas. The indirect hydrogenation process includes the generation of syngas from the CO₂ and low carbon intensity hydrogen, which is further reacted over a Fischer-Tropsch catalyst to yield the hydrocarbons with a carbon distribution between C1 and C33+. The indirect hydrogenation also generates gases and an aqueous product.

Next, the stream undergoes a primary separation step and is divided into the gases (CO₂, CO, H₂, and C1-C4), the hydrocarbons (C5-C23+), and the aqueous phase. The gases resulting from the primary separation step are divided. The CO₂, CO, and H₂ are recycled, and the C1 to C4 gases are used in other areas of the refinery. For example, the C1-C2 gases can be used as fuels, and the C3 and C4 gases can serve as feedstocks to generate low carbon intensity (CI) alkylate. The aqueous phase is disposed of using the existing water treatment plant at the refinery. There is no need for a costly separation process for this CO₂ hydrogenation, thus decreasing cost and increasing profitability.

Like the process of the fifth embodiment, the hydrocarbons resulting from the primary separation step are divided into naphtha, diesel, and vacuum gas oil (VGO). The naphtha includes C5-C10 hydrocarbons and the diesel includes C15-C23 hydrocarbons. A stream containing naphtha and diesel can be fed to a coker distillation step and hydro-treatment step. The vacuum gas oil includes C23+ hydrocarbons and is fed to a fluid catalytic cracker or another hydrocracker available within the refinery to produce motor gasoline (MOGAS), jet fuel, diesel and/or another fuel. All of the resulting streams have a low carbon intensity (CI) and can be used as fuel components. The carbon intensity (CI) of the facility is decreased by using sustainable CO₂ as a carbon source, and high-value products are generated. As indicated above, the process according to the sixth embodiment, like the other embodiments described above, is able to use existing petroleum refinery facilities, which significantly reduces capital and operating expenses.

The specific embodiments described above have been shown by way of example, and it should be understood that these embodiments may be susceptible to various modifications and alternative forms, and can also be used in any appropriate combination. It should be further understood that the claims are not intended to be limited to the particular forms disclosed, but rather to cover all modifications, equivalents, and alternatives falling within the spirit and scope of this disclosure.

1. A process for producing fuel or a fuel component, comprising the steps of:
hydrogenating carbon dioxide (CO₂) at a downstream facility used to produce oil or gas;
the hydrogenation step producing gases, hydrocarbons, and an aqueous phase; and
separating, processing, and/or recycling the gases, the hydrocarbons, and the aqueous phase at the downstream facility.

2. The process according to claim 1, wherein the CO₂ is obtained from a source located at the downstream facility.

3. The process according to claim 1 including feeding the aqueous phase to a water treatment plant at the downstream facility for disposal.

4. The process according to claim 1 including processing and recycling the gases produced from the hydrogenation step at the downstream facility.

5. The process according to claim 1, wherein the hydrogenation step includes indirect hydrogenation of the carbon dioxide according to Equation 1:
CO₂+H₂→Syngas (H₂/CO)→Hydrocarbons+H₂O (1).

6. The process according to claim 1, wherein the hydrogenation step includes direct hydrogenation of the carbon dioxide according to Equation 2:
CO₂+H₂→Hydrocarbons+H₂O (2).

7. The process according to claim 1 including feeding the hydrocarbons to a crude distillation unit and distilling the hydrocarbons to produce a feedstock.

8. The process according to claim 1 including dividing the hydrocarbons into naphtha, jet fuel, diesel, vacuum gas oil (VGO), and/or waxes.

9. The process according to claim 8, wherein the naphtha includes C5-C10 hydrocarbons, and including the step of feeding the naphtha to a naphtha hydrotreater to produce a gasoline component.

10. The process according to claim 8, wherein the jet fuel includes C10-C15 hydrocarbons, and feeding the jet fuel to a jet fuel hydrotreater to produce a jet fuel component.

11. The process according to claim 8, wherein the diesel includes C15-C23 hydrocarbons, and feeding the diesel to a diesel hydrotreater to produce a diesel component.

12. The process according to claim 8, wherein the vacuum gas oil includes C23+ hydrocarbons, and feeding the vacuum gas oil to a fluid catalytic cracker or another hydrocracker within the downstream facility to produce motor gasoline (MOGAS), jet fuel, diesel and/or fuel gas.

13. The process according to claim 8, wherein the vacuum gas oil includes C23+ hydrocarbons, the wax includes C19-C24 hydrocarbons with the formula CₙH₂ₙ₊₂, and processing the vacuum gas oil and the wax in a fluid catalytic cracker or another hydrocracker within the downstream facility to produce motor gasoline (MOGAS), jet fuel, diesel and/or fuel gas.

14. The process according to claim 8, wherein the naphtha includes C5-C10 hydrocarbons, the diesel includes C15-C23 hydrocarbons, and including the steps of distilling the naphtha and diesel in a coker distillation unit, and hydro-treating the naphtha and diesel.

15. The process according to claim 1, wherein the downstream facility is a petroleum refinery.

16. The process according to claim 1, wherein the source of the CO₂ is flue gas.

17. The process according to claim 1, wherein products of the hydrogenation have a carbon intensity of not greater than 80 gCO₂e/MJ.

18. The process according to claim 1, wherein the gases include CO₂, CO, and H₂.

19. The process according to claim 1, wherein the hydrogenation step includes reacting the CO₂ with hydrogen, the hydrogen having a carbon intensity of not greater than 50 gCO₂e/MJ.

20. The process according to claim 1 including processing the gases using equipment at the downstream facility; and/or recycling the gases and using the gases in the downstream facility.
